(19) United States
(12) Patent Application Publication
(10) Pub. No.: US 2015/0080310 A1
(43) Pub. Date: Mar. 19, 2015

(54) BIOCOMPATIBLE COMPOSITION

(71) Applicant: NMI NATURWISSENSCHAFTLICHES UND MEDIZINISCHES INSTITUT AN DER UNIVERSITAET TUEBINGEN, Reutlingen (DE)

(72) Inventors: Larissa Lehmann, Kissing (DE); Elke Rist, Eningen (DE); Beate Scholz, Dossenheim (DE); Burkhard Schlosshauer, Tuebingen (DE); Juergen Mollenhauer, Reutlingen (DE); Christoph Gaissmaier, Kusterdingen-Maehringen (DE)

(21) Appl. No.: 14/550,731

(22) Filed: Nov. 21, 2014

Related U.S. Application Data
(63) Continuation of application No. PCT/EP2013/059698, filed on May 10, 2013.

(30) Foreign Application Priority Data
May 25, 2012 (DE) 10 2012 104 530.5

(51) Int. Cl. A61K 47/48; A61K 38/38

(52) U.S. Cl. CPC A61K 47/48784; A61K 38/385; A61K 47/48023; A61K 47/4823; A61K 47/48176
USPC 514/15.2

(57) ABSTRACT

A biocompatible composition which comprises crosslinkable serum albumin, crosslinkable serum protein and/or crosslinkable derivatives derived therefrom, and which is polymerizable to give a hydrogel-forming material, is used in a method for the prevention of pathological adhesions.

### RELATED APPLICATION

This is a continuation application of co-pending international patent application PCT/EP2013/059698, filed May 10, 2013 and designating the United States, which was published in English as WO 2013/174661 A1, and claims priority to German patent application DE 10 2012 104 530.5, filed May 25, 2012, which is incorporated herein by reference.

### BACKGROUND OF THE INVENTION

1. Field of the Invention

The present invention relates to a biocompatible composition which comprises crosslinkable serum albumin, crosslinkable serum protein and/or crosslinkable derivatives derived therefrom, and which is polymerizable to give a hydrogel-forming material.

The present invention further relates to a hydrogel-forming material which was obtained by polymerizing a biocompatible composition of this type.

2. Related Prior Art

A biocompatible composition of this type and a hydrogel-forming material of this type are described in DE 10 2007 034 580 A1.

According to this document, the composition and the material polymerized therefrom are used for culturing cells and tissues.

DE 10 2008 008 071 A1 discloses that the material known from DE 10 2007 034 580 A1 mentioned at the outset, when it is based on crosslinkable serum albumin or serum protein, is used as injectable biocompatible composition in order to support, for example, cartilage growth.

Finally, it is known from DE 10 2009 051 575 A1 that hydrogels of this type prevent the sprouting of blood vessels, that is have an anti-angiogenic effect.

The known biocompatible composition is polymerized by adding an SH crosslinker to form a hydrogel-forming material which is based on the hydrophilic, for example albumin-based, components.

A “hydrogel”, in the context of the present invention, is taken to mean a water-containing, but itself water-insoluble, polymer, the molecules of which are chemically linked to form a three-dimensional matrix. Owing to the hydrophilic components incorporated, hydrogels swell in water, increasing in volume, without losing in the process their material cohesion.

In the present application, the expressions “composition” and “material” are used, wherein “composition” is used for the polymerizable precursor component of the completely polymerized material. “Material” or “gel”, in contrast, represent the composition polymerized with the aid of a crosslinker Nevertheless, of course, these expressions cannot be completely separated from each other, since the composition and the material in effect mean the same object, especially since the transition from the composition to the material proceeds continuously. In this case “gel” is taken to mean the semi-solid state of the material which is present in the form of a three-dimensional polymerized network.

It is known from DE 10 2009 051 575 mentioned at the outset that the composition applied or injected in this manner or the completely polymerized material simultaneously serves as tissue substitute or implant, and inhibits the adhesion and proliferation of endothelial cells thereon. As a result, the de novo formation of blood vessels and also swelling and thickening of the tissue into which the composition is introduced for replacing diseased or deficient tissue are avoided, and at the same time the deficient or diseased tissue is replaced by resorption of the material.

In other words, in the known use, the composition and the material are used in order firstly to inhibit angiogenesis specifically, and secondly to promote, by prior introduction into the composition/the material, specifically the growth of other cells which do not participate in angiogenesis.

The composition can in this case also be first polymerized in situ, which means the composition can be injected in the liquid state into the site where the tissue substitute or the tissue support is to take place, and not until then polymerized to completion at this site. As a result, for the known therapeutic treatment, only a minimally invasive medical intervention is necessary.

Secondly, the composition can also be polymerized to completion before introduction into the body of a patient, and then implanted, for example as hydrogel, via a surgical intervention.

The known composition is biocompatible and resorbable, because it is based on serum albumin and/or serum protein, and the crosslinked albumin dissolves within a certain time period after introduction into the patient.

Exemplary methods for producing the composition may be found in the previously mentioned DE 10 2008 008 071 A, the contents of which in this regard are explicitly incorporated herein by reference.

### SUMMARY OF THE INVENTION

The inventors of the present application have now discovered that such a biocompatible composition which comprises crosslinkable serum albumin, crosslinkable serum protein and/or crosslinkable derivatives derived therefrom, and which is polymerizable to give a hydrogel-forming material, is suitable for use in the prevention of pathological adhesions.

In first experiments, the inventors have found that a hydrogel of this type, originally designed for cartilage regeneration, not only prevents angiogenesis by endothelial cells, but also scar formation by fibroblasts. In a first key experiment with abdominal traumata in rats, it was found that the known hydrogel is an efficient adhesion barrier which can prevent pathological accretions (adhesions) between wounded tissue surfaces, especially in the abdominal cavity.

Such adhesion barriers are used in the prior art in order to restrict, or preferably entirely prevent, the growth in the form of a pathological scar formation between wounded tissue surfaces.

Connective tissue scar structures are first a natural and also necessary endogenious wound treatment. However, problems occur when tissue functions are disturbed by scar formation. Pathological consequences comprise, for example, the impairment of nerve pathways running in the wound region, which can lead to the development of unbearable pain, in the abdominal region the blockade of nutrient flow, or the fusion of vertebrae.

Pathological scar formations can occur in this case, in principle, in the entire body, by tumours, radiation treatments, wounds and surgical interventions. These problems occur, especially, in human patients, but also in other mammals.

Purely surgical treatment of pathological adhesions of this type frequently only solves the problem in the short term until new scarring forms.

In order to counteract pathological accretions or scar formations of this type, therefore, in the prior art, implants are known which are introduced as adhesion barriers between the tissue regions which are not to scar or fuse during a healing process.

According to knowledge of the inventors, two resorb able membranes are market dominating, inter alia, for the abdominal region (Interceed® and Seprafilm®) and one for spinal column therapy (Duragen®). The base materials for these products are collagen and hyaluronic acid.

Two further products have had to be withdrawn from the market, inter alia, because of toxic side effects.

Furthermore, e.g. viscous solutions are available on the market as spacers of contested activity.

Although the biocompatible composition used according to the invention and the hydrogel-forming material polymerized therefrom has previously qualified as a matrix for the ingrowth of cells, the inventors of the present application have surprisingly found that the hydrogel-forming material is suitable as an adhesion barrier, and therefore prevents the accretion of tissue surfaces when it is applied between these tissue surfaces.

According to one embodiment the serum albumin, the serum protein or the derivatives derived therefrom are functionalized by groups which are selected from maleimide, vinylsulphonic, acrylate, alkyl halide, azirine, pyridyl, thionitrobenzene acid groups, or arylating groups.

“Functionality” or “functionalizing” in the present context is understood to mean any—finished—process by which the composition is given a function which it does not normally possess—for example by addition of groups to the crosslinkable components of the composition.

By functionalizing with maleimide groups, a particularly good crosslinking of the polymer can be ensured.

In the context of the present application, derivatives of serum albumin or serum protein are taken to mean those polymers which, proceeding from serum albumin or serum protein, are generated by derivatization, which is taken to mean, for example, covalent attachment of further biological factors, changes in charge or polarization state and also of protein structure.

In this manner, the crosslinkable components can be modified in their chemical and physical properties and also in their biological activity in such a manner that they can be stored isolated from the crosslinker sufficiently long and, on combination with the crosslinker, have the desired properties and effects.

The composition to be used according to the invention and/or the polymerized hydrogel-forming material based thereon according to one embodiment comprise serum albumin and/or serum proteins that are obtained from any mammal, or can be used correspondingly for any mammal, wherein human, bovine, sheep, rabbit serum albumin are preferred, wherein the use according to the invention is in one embodiment in humans, using a material based on human serum albumin.

In addition, it is advantageous in this case that the base material for the composition used according to the invention is variable, thus, firstly, commercially available albumin, for example human albumin, purified or recombinantly produced, can be used, and also allogenous or autologous serum.

The composition may comprise a pharmacologically active agent which is preferably selected from at least one of the following: an antibiotic, a cytostatic, an anti-inflammatory, a metabolism hormone, agents for gene therapy, growth hormones, differentiation or modulation factors, immunosuppressants immunostimulating substances, nucleic acids, apoptosis-inducing agents, adhesion-inducing or inhibiting agents, receptor agonists and receptor antagonists, or mixtures thereof.

Of course, the use according to the invention can also proceed in interaction with biologically or pharmaceutically active substances. “Biologically active substance” and “pharmaceutically active substance” shall mean here any natural or synthetic substance which can have either a biological or pharmaceutical effect on cells or tissue, or can effect the reactions on or in cells.

This effect can be limited here to certain cells and certain conditions, without the substance losing its biologically or pharmaceutically active meaning. The chemical nature of the substances useable in the present case is not limited here to a specific class (of compounds), but can rather include any natural and synthetic substance which in its natural state and/or in modified form has any effect on biological cells.

According to one object, the composition is used in situ for polymerization to give a hydrogel-forming material.

Here it is advantageous that the composition is not polymerized to completion until it has been introduced into the body of the patient at the site that is to be protected.

The composition in this case can be used in injectable form or in sprayable form, wherein it is used in one embodiment in a minimally invasive manner, although it is also possible to use it in connection with a surgical intervention.

The composition in this case is mixed with a crosslinker, for example immediately before application thereof into the body, and then this mixture is either introduced into the body of the patient as a liquid or as a spray, in such a manner that the polymerization proceeds only in situ. However, it is also envisaged to conduct the composition and the crosslinker separately to the site in the body of the human or animal patient that is to be protected, and to mix them there.

In the case of the hydrogel-forming material which was obtained by polymerizing the described composition, it is particularly preferred if it was obtained by polymerizing the composition with a crosslinker, wherein the crosslinker is preferably an SH crosslinker

In this case, in particular the crosslinker bis-thio polyethylene glycol comes into use, which at both ends carries an SH group. In addition to bis-thio-PEG, as crosslinkers, generally other substances also come into use which carry SH groups, in particular polymers, and, for example, dithio-PEG or SH-modified dextran, SH-modified polyvinyl alcohol, SH-modified polyvinylpyrrolidone.

This hydrogel-forming matrix can be generated immediately before surgical or minimally invasive application and then used as a spray, as an implant, as a liquid, as a plug or as a gel film. It is therefore introduced into the body of a patient after it has been produced.

The material polymerizes to completion in this case either before application, during application or else after application.

Of course, during an implantation of the completely polymerized hydrogel, a somewhat solid consistency of the hydrogel is preferred which permits or facilitates practical handling of the hydrogel. The degree of solidity or fluid properties of the hydrogel or of the material in this case can be set via by the degree of crosslinking, wherein the hydrogel or the material is the more solid the more it is crosslinked The fluid properties of a gel are thus between those of a liquid and those of a solid.

The present invention also relates to a kit having a first container which comprises the described composition and having a second container which comprises a crosslinker for the composition for use in the prevention of pathological adhesions, wherein the kit is in one embodiment suitable for use in the in situ generation of a hydrogel-forming material.

When both the composition having the generally functionalized serum albumin, serum protein or the derivatives derived therefrom is provided in a first container and a crosslinker suitable therefor is provided in a second container, the composition and the crosslinker can be matched to one another in such a manner that the hydrogel suitable for the respective desired treatment is formed.

In this case the rate of polymerization, the viscosity, the resorption kinetics etc. can be adjusted in such a manner that the components present in the two containers of the kit are separately or jointly sprayable or injectable as a liquid.

In particular, the advantage is linked with the use according to the invention of the hydrogel that is known per se that, in the liquid state, it can be applied to traumatized and intact tissue surfaces or in tissues which are surgically cleared.

The hydrogel, however, can also be used without problems in a minimally invasive manner, for example as a liquid or as a spray.

The resultant gel adapts in each case immediately to non-uniform tissue surfaces, wherein it is also formed on moist tissue surfaces without running significantly.

As a result, very thin hydrogel layers can be formed on the tissue surfaces, since even layer thicknesses of less than 1 mm are sufficient in order to prevent pathological adhesions.

A further advantage is considered that the hydrogel layers are rapidly resorbed, generally within a residence time of about seven days, wherein the hydrogel is not encapsulated, that is does not lead to secondary fibroses.

In addition, the hydrogel is outstandingly compatible and does not trigger any inflammations or pathological blood vessel formation.

Compared with the adhesion barriers known from the prior art and mentioned at the outset, the hydrogel used according to the invention therefore has, in particular, the advantages that it is highly compatible, does not initiate any inflammations, does not cause scarring because it is degraded, is anti-angiogenic and, according to the latest knowledge, also acts in an anti-neuritotrophic manner, that is to say it does not cause any nerve sprouting.

In addition, the hydrogel is simple to handle, because both components can be first mixed shortly before application, wherein the two components are either injected or sprayed on. For spraying or injection, the components can be combined shortly before or at the site in the body that is to be protected, or else distally to this site.

Certain requirements must be made of an adhesion barrier on the basis of use by the surgeon, the site of use and the functionality. Firstly, a barrier should be biocompatible, resorbable and non-immunogenic. It must be able to keep wounded surfaces apart from one another and to prevent fibrin strand formation between the damaged tissues. For clinical use, in addition, it must be simple to handle and apply, and also suitable for minimally invasive interventions.

In addition, a barrier should be functional over the critical time frame of seven days, which means that it must remain at the site and in position and still must not be completely dissolved.

Application should be possible without stitching, in order not to provoke any new adhesions.

All these requirements are met outstandingly by the hydrogel described here.

Further advantages result from the description and the accompanying drawing.

Of course, the features that are abovementioned and are still to be explained hereinafter are not only useable in the combinations respectively stated, but also in other combinations or alone, without departing from the scope of the present invention.

### BRIEF DESCRIPTION OF THE DRAWINGS

Embodiments of the invention are shown in the drawing and are described in more detail in the description hereinafter. In the drawing:

FIG. 1 shows a kit having a first container, in which a composition is situated as first component which comprises crosslinkable serum albumin, and a second container, in which, as second component, a crosslinker is present, wherein after combination of these two components, the composition is polymerized to form a hydrogel;

FIG. 2 shows an image of an abdominal cavity of a rat, seven days after adhesion formation prevented postoperatively;

FIG. 3 shows a positive control to the experiment according to FIG. 2; and

FIG. 4 shows a diagram giving the qualitative assessment of four animals treated according to the invention and also eight control animals.

### DESCRIPTION OF PREFERRED EMBODIMENTS

### EXAMPLES

### A) Preparation of Maleimide-Modified Serum Albumin

As an example for preparing the composition used according to the invention, hereinafter the production of maleimide-modified serum albumin is described.

250 mg of human, rabbit or sheep serum albumin (Sigma-Aldrich) were dissolved in 5 ml of 1M Na borate (pH 8.2). Thereto were added 75 μl of a 260 mM N-maleoyl-β-alanine (Sigma-Aldrich Cat. No. 63285) solution in PBS/Na borate (pH 8.2) (1:1), and the mixture was incubated for 90 min at room temperature. 106 mg of 3-maleimidopropionic acid N-hydroxysuccinimide ester (SMP, Obiter Research, Urbana, Ill., USA) dissolved in 950 μl of dimethylformamide (DMF). Insoluble material was removed by centrifugation. 500 μl of the supernatant were added to the albumin solution which was then incubated for 60 min at room temperature. Thereafter, 500 μl of 3M sodium acetate (pH 4.7) were added thereto and dialyzed three times against 1 liter of PBS on ice. The dialysate was then concentrated by ultrafiltration (YM-3 membrane, Millipore) to a volume of 3.5 ml, filter-sterilized, and stored at −80° C.

### B) Preparation of the Hydrogel

The serum albumin/protein thus functionalized can be polymerized by adding SH crosslinkers. In this case, in particular the crosslinker bis-thio polyethylene glycol comes into consideration, which has an SH group at both ends. In addition to bis-thio-PEG, crosslinkers which come into consideration are generally substances which carry SH groups, in particular polymers, and, for example, dithio-PEG, or SH-modified dextran, SH-modified polyvinyl alcohol, SH-modified polyvinylpyrrolidone, etc.

Bis-thio-PEG is commercially available; the crosslinker used was that having a molar mass of 10 000 g/mol. If the molar mass is lower, gel formation is reduced, at higher masses the gel gelates too rapidly, which makes sufficient mixing of the substances impossible. The best gel formation is achieved when SH groups of the crosslinker and maleimide groups of the albumin are present in equimolar concentrations. A final concentration of 3 mM maleimide and SH groups was used in each case in the gel.

### C) Preparation of the Two Components

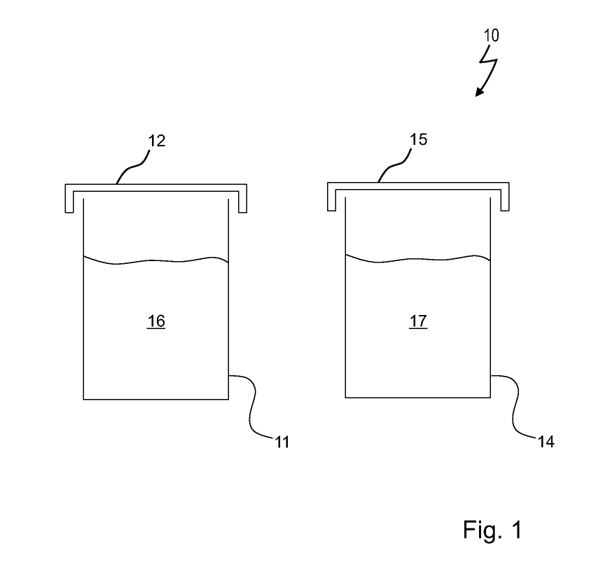

FIG. 1 shows a kit 10 which comprises a first container 11 having a closing lid 12 and a second container 14 having a closing lid 15. In the first container 11 there is situated the composition according to Example A) designated by 16, and in the second container 14 there is situated the crosslinker 17 from Example B) designated by 17.

Components 16, 17 can be stored in this manner for a relatively long time and applied separately, or after combination by spraying or injection, or after polymerization as liquid, spray or gel.

### D) Induced Abdominal Wound

Once it was already known for the known hydrogel that it is nontoxic and is resorbed within two weeks or earlier, the function as an adhesion barrier as such had to be tested.

Such experiments are based to date only on the use of animal models. The animals are subjected in this case to an adhesion-inducing operation, wherein here different operation techniques are established.

In such experiments, frequently large numbers of animals are used, since, firstly, the triggering of adhesions in the control animals does not function in a standardized manner, and secondly, mechanical control had not been previously standardized.

In a pilot experiment, in a rat induced abdominal injuries were caused, by mechanically injuring mutually opposite tissue surfaces of the peritoneum and of the intestine of a rat.

Within a few days, in the case of animals that were not treated further, adhesions reliably formed between the two tissue surfaces, as also occur after human abdominal cavity operations.

For testing the novel adhesion bather, a hydrogel, as described above, was introduced between the two wounded tissues. At various time points after implantation, the tissues were then analyzed macroscopically and histologically.

Furthermore, the strength of the adhesion was determined semi-quantitatively using a tension experiment, as was described for the first time in the dissertation by Ms Larissa Grupp “Untersuchungen von Reaktionen auf gelatinebasierte Implantate in verschiedenen Organsystemen” [“Studies of Reactions to Gelatin-based Implants in various Organ Systems”].

In the experiments carried out here, the rats were first anaesthetized with a mixture of ketamine and xylazine. After absence of the reflexes, the animal was put on its back, and the ventral side was shaved.

For opening the abdominal cavity, first an approximately 4 cm long skin incision was made at the height of the translucent Linea alba. The Linea alba was then cut with a 4 cm long incision.

For the pilot experiment described here for testing the hydrogel as adhesion barrier, four animals were operated on. The hydrogel was applied unilaterally to the intestinal surface. The analysis was performed seven days later.

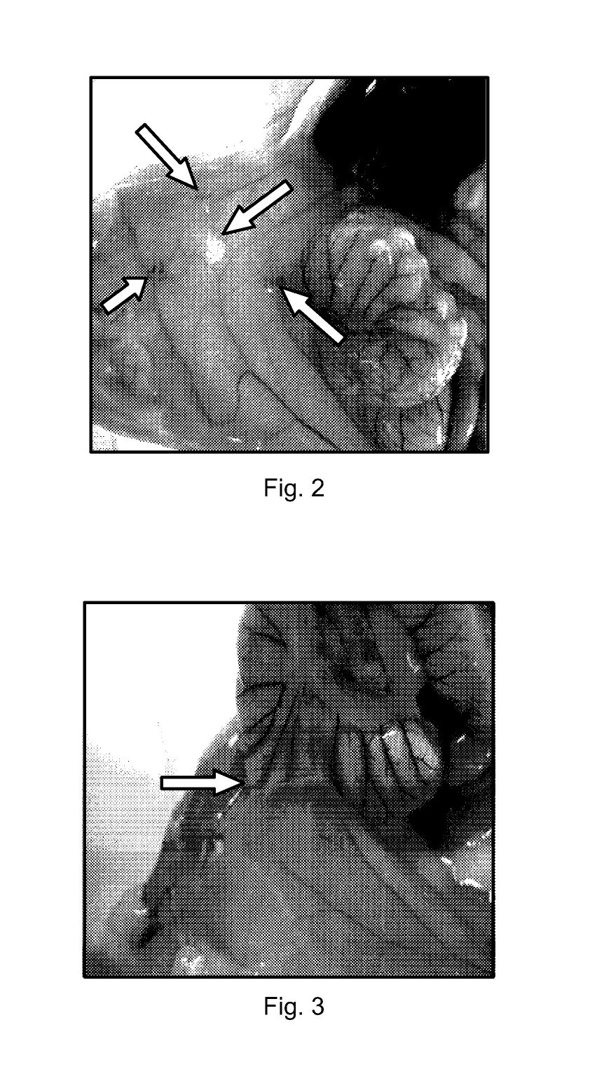

From FIG. 2, it may be seen that after seven days postoperative, adhesion formation was prevented by application of the hydrogel.

In comparison therewith, FIG. 3 shows that after seven days postoperative, intestine and peritoneum had adhered together, if no hydrogel had been applied.

In FIG. 3, it may be seen that between the tissues, new scar tissue had formed; this is the whitish tissue between intestine and peritoneum indicated by an arrow.

In contrast thereto, FIG. 2 shows that the formation of an adhesion and also angiogenesis and inflammation were completely prevented.

For this purpose, the hydrogel (a small residue appears as a light spot and is indicated by a black arrowhead) was placed on the peritoneum centrally to the three arrows (scar material residues). The hydrogel layer according to the invention had been applied between the experimentally injured intestine (folded to the right, broad arrowhead) and the peritoneum.

In the qualitative assessment, the tissue surfaces were pulled apart in a controlled manner and the force necessary for this was measured. With the hydrogel between the injured tissues, in 75% of the animals that had been operated on no adhesion occurred, and in the fourth animal the gel had obviously been misplaced.

The exposed tissue surfaces showed no redness, the hydrogel was not surrounded by tissue or even encapsulated. Therefore, no inflammatory reaction occurred.

Eight control animals, in contrast, showed adhesions at 100%.

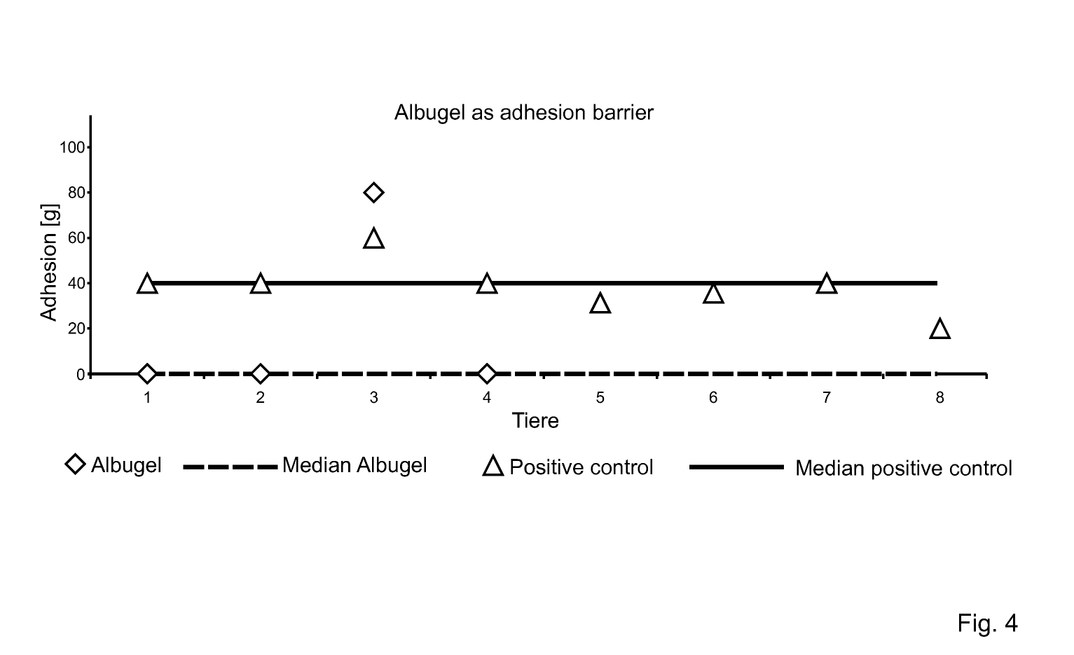

In the qualitative assessment, the tensile force is shown in grams. FIG. 4 shows a corresponding diagram for four hydrogel-treated animals and eight control animals.

The treated animals are indicated by rectangles standing on the point, and the control animals are indicated by triangles standing on the base.

It may be seen that in the control animals, on average, an adhesion force of 40 g had to be overcome in order to separate the injured tissues from one another, whereas in three of the four treated animals the tissue was able to be separated without any force being exerted.

Therefore, what is claimed is:

1. A method for preventing pathological adhesions, comprising the step of administering to a subject in need thereof a biocompatible composition, said biocompatible composition comprising a crosslinkable substance selected from the group consisting of crosslinkable serum albumin, crosslinkable serum protein, crosslinkable derivatives derived from crosslinkable serum albumin, and crosslinkable derivatives derived from crosslinkable serum protein, which crosslinkable substance is polymerizable to give a hydrogel-forming material.

2. The method of claim 1, wherein the crosslinkable substance is functionalized by groups which are selected from among maleimide, vinylsulphonic, acrylate, alkyl halide, azirine, pyridyl, thionitrobenzene acid groups, or arylating groups.

3. The method of claim 1, wherein the serum albumin and the serum protein are human serum albumin and human serum protein.

4. The method of claim 1, wherein the biocompatible composition comprises a pharmacologically active agent which is selected from at least one of the following: an antibiotic, a cytostatic, an anti-inflammatory, a metabolism hormone, agents for gene therapy, growth hormones, differentiation or modulation factors, immunosuppressants, immunostimulating substances, nucleic acids, apoptosis-inducing agents, adhesion-inducing or adhesion-inhibiting agents, receptor agonists and receptor antagonists, and mixtures thereof.

5. The method of claim 1, wherein biocompatible composition is polymerized in situ to give a hydrogel-forming material.

6. The method of claim 5, wherein the biocompatible composition is administered in injectable form.

7. The method of claim 5, wherein the biocompatible composition is administered in in sprayable form.

8. The method of claim 5, wherein the biocompatible composition is administered in in a minimally invasive manner.

9. The method of claim 5, wherein the biocompatible composition is administered surgically.

10. The method of claim 5, wherein the biocompatible composition is administered as a liquid.

11. The method of claim 5, wherein the biocompatible composition is administered as a gel film.

12. The method of claim 5, wherein the biocompatible composition is administered as a plug.

13. The method of claim 5, wherein the biocompatible composition is administered as an implant

14. The method of claim 1, wherein the biocompatible composition is polymerized with a crosslinker to give said hydrogel-forming material.

15. The method of claim 14, wherein the biocompatible composition is polymerized with an SH crosslinker to give said hydrogel-forming material.

16. The method of claim 14, wherein the biocompatible composition is polymerized prior to administering it to said subject.

17. A method for preventing pathological adhesions in human or animal patients, comprising the step of administering a hydrogel-forming material to a site to be treated in the body of the patient, wherein said hydrogel-forming material is obtained by polymerizing a biocompatible composition, said biocompatible composition comprising a crosslinkable substance selected from the group consisting of crosslinkable serum albumin, crosslinkable serum protein, crosslinkable derivatives derived from crosslinkable serum albumin, and crosslinkable derivatives derived from crosslinkable serum protein.

18. The method of claim 17, wherein the hydrogel-forming material is obtained by polymerizing the composition with a crosslinker.

19. The method of claim 18, wherein the hydrogel-forming material is obtained by polymerizing the composition with an SH crosslinker.

20. The method of claim 17, wherein the hydrogel-forming material is prepared before introduction into the body of the patient.

21. The method of claim 17, wherein the hydrogel-forming material is sprayed onto said site.

22. The method of claim 17, wherein the hydrogel-forming material is implanted into said site as a liquid, a gel film or a plug.

23. The method of claim 18, wherein the hydrogel-forming material is prepared during introduction into the body of the patient by applying said biocompatible composition and said crosslinker separately to the site, and combining them at the site or prior to reaching the site.

24. The method of claim 18, wherein the crosslinkable substance is functionalized by groups which are selected from among maleimide, vinylsulphonic, acrylate, alkyl halide, azirine, pyridyl, thionitrobenzene acid groups, or arylating groups.

25. A kit including a first container which includes a biocompatible composition, said biocompatible composition comprising a crosslinkable substance selected from the group consisting of crosslinkable serum albumin, crosslinkable serum protein, crosslinkable derivatives derived from crosslinkable serum albumin, and crosslinkable derivatives derived from crosslinkable serum protein, and a second container which comprises a crosslinker for the biocompatible composition, for use in the prevention of pathological accretions.

26. The kit of claim 25 for use in the in situ generation of a hydrogel-forming material.

27. A method for preventing pathological adhesions in human or animal patients, comprising the step of administering a hydrogel-forming material to a site to be treated in the body of the patient, wherein said hydrogel-forming material is obtained by polymerizing a biocompatible composition with a crosslinker, said biocompatible composition comprising a crosslinkable substance selected from the group consisting of crosslinkable serum albumin, crosslinkable serum protein, crosslinkable derivatives derived from crosslinkable serum albumin, and crosslinkable derivatives derived from crosslinkable serum protein, said crosslinkable substance being functionalized by groups which are selected from among maleimide, vinylsulphonic, acrylate, alkyl halide, azirine, pyridyl, thionitrobenzene acid groups, or arylating groups.

28. The method of claim 27, wherein the biocompatible composition comprises serum albumin functionalized by maleimide or serum protein functionalized by maleimide, and said hydrogel-forming material is obtained by polymerizing said biocompatible composition with an SH crosslinker.
